(19) United States
(12) Patent Application Publication
(10) Pub. No.: US 2013/0012878 A1
(43) Pub. Date: Jan. 10, 2013

(54) PROGRAMMING MEDICAL PUMPS WITH ELECTRONIC STANDING ORDER TEMPLATE

(76) Inventor: Michael L. Blomquist, Blaine, MN (US)

(21) Appl. No.: 13/619,740

(22) Filed: Sep. 14, 2012

Related U.S. Application Data
(62) Division of application No. 11/003,147, filed on Dec. 3, 2004.
(60) Provisional application No. 60/526,810, filed on Dec. 4, 2003.

(51) Int. Cl. A61M 5/142; G06F 17/30

(52) U.S. Cl. 604/151

(57) ABSTRACT

A method of programming a medical pump, the method comprising: selecting a therapy from a memory; upon selection of a therapy, selecting a therapeutic agent for delivery; and generating a standing order, the standing order including data for controlling operation of the medical pump.

### RELATED APPLICATIONS

This application is a division of application Ser. No. 11/003,147 filed Dec. 3, 2004, which claims the benefit of U.S. Provisional Application No. 60/526,810 filed Dec. 4, 2003, each of which is hereby fully incorporated herein by reference.

### TECHNICAL FIELD

The present invention related to medical pumps, and more particularly to programming medical pumps with electronic standing order templates.

### BACKGROUND

In order to improve patient safety and overall efficiencies, hospitals oftentimes create standing orders for various types of drug delivery. These standing orders are usually for a therapy, not a drug. For example, standing orders for patient controlled analgesia oftentimes allow the doctor to choose from a list of drugs indicated for the therapy. Once the doctor has chosen a particular drug he/she fills in the blanks with the specific delivery parameters for that drug. The standing orders sheet is a paper worksheet that provides ranges and starting values along with other clinically pertinent information to aid the doctor in properly prescribing the particular therapy and the nurse in properly delivering and monitoring the therapy.

### SUMMARY

In general terms, the present invention is directed to programming a medical pump by selecting a therapy from a memory.

One aspect of the present invention is a computer readable medium in a computer apparatus. The computer readable medium contains a database, the database programmed to including a plurality of therapies, a plurality of therapeutic agents, at least one of the therapeutic agents being linked to at least one therapy, and a plurality of standing orders, each standing order including data for controlling operation of a programmable medical pump.

Another aspect of the present invention is a computer readable medium in a programmable medical pump. The computer readable medium containing a database, the database programmed to including a plurality of therapies, a plurality of therapeutic agents, at least one of the therapeutic agents being linked to at least one therapy, and a plurality of standing orders, each standing order including data for controlling operation of a programmable medical pump.

Another aspect of the present invention is a programmable pump for delivering fluid to a patient. The pump comprises a pump mechanism and a programmable circuit arranged to control the pump mechanism. Memory is in electrical communication with the programmable circuit. The memory is configured to store one or more therapies, one or more therapeutic agents, and links between at least one of the therapies and at least one of the therapeutic agents.

Another aspect of the claimed invention is an apparatus for programming a medical pump. The apparatus comprises memory loaded with a database, the database including one or more therapies, one or more therapeutic agents, and one or more standing order templates. A data output is configured for data communication with a programmable medical pump. A processor is in electrical communication with the memory and the data output. The processor is configured to select a therapy from the memory; select a therapeutic agent from the memory after selection of a therapy; and generate a standing order corresponding to the selected therapy and the selected therapeutic agent.

Yet another aspect of the claimed invention is a method of programming a medical pump, the method comprising: selecting a therapy from a memory; upon selection of a therapy, selecting a therapeutic agent for delivery; and generating a standing order, the standing order including data for controlling operation of the medical pump.

### BRIEF DESCRIPTION OF THE DRAWINGS

FIG. 1 is a diagram of a system of one possible embodiment of the present invention.

FIG. 2 is a table illustrating electronic standing orders.

FIG. 3 is a flowchart of operations for one possible embodiment of the present invention.

FIG. 4 is a flowchart of operations for another possible embodiment of the present invention.

### DETAILED DESCRIPTION

A preferred embodiment of the invention will be described in detail with reference to the drawings, wherein like reference numerals represent like parts and assemblies throughout the several views. Reference to the preferred embodiment does not limit the scope of the present invention.

Additionally, the logical operations of the various embodiments of the invention described herein are implemented as: (1) a sequence of computer implemented steps running on a computing system; and/or (2) interconnected machine modules within the computing system. The implementation used is a matter of choice dependent on the performance requirements of the desk-top computer, server, mobile computing platform, and medical pump. Accordingly, the logical operations making up the embodiments of the invention described herein are referred to alternatively as operations, steps, or modules.

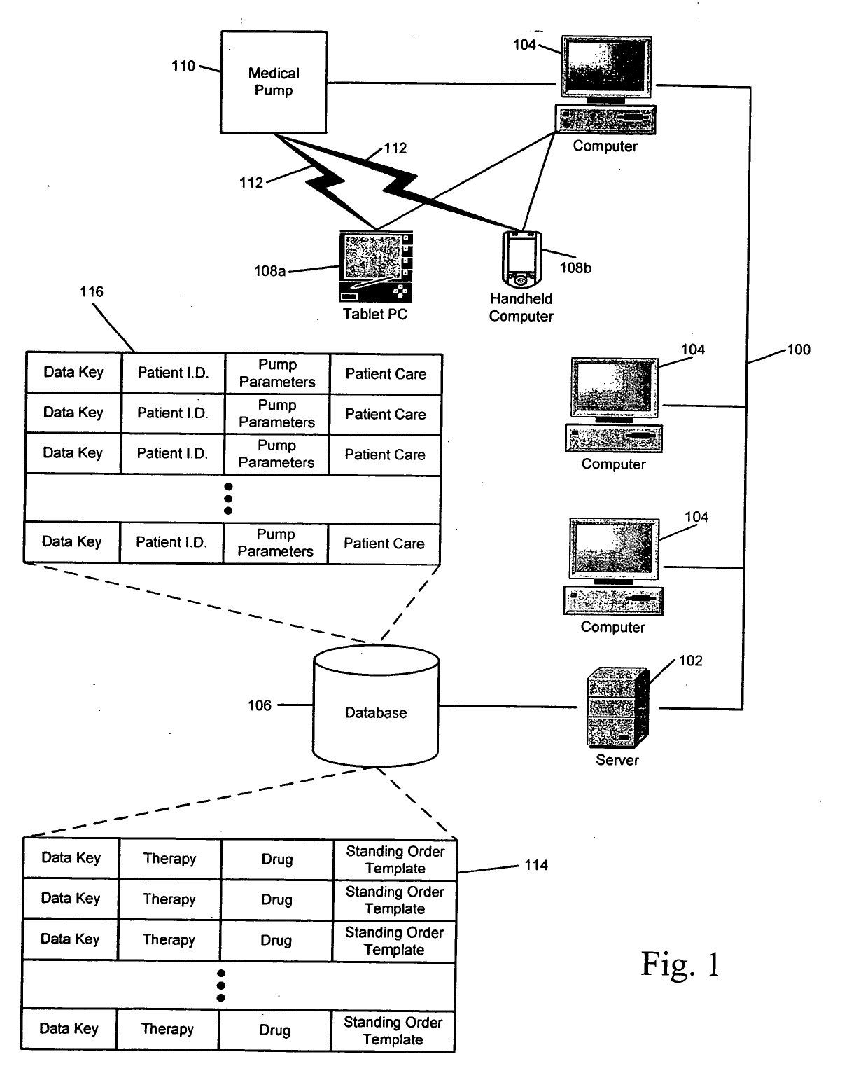

Referring to FIG. 1, one possible embodiment includes a computer network 100 such as a local area network, to which a server 102 and a plurality of computers 104 are connected. Memory storing one or more databases 106 is in data communication with the server 102. In one possible embodiment, the one or more databases 106 includes a standing order database 114 and a patient database 116. Additionally, a mobile computing platform 108 such as a personal data device (PDA) 108a or tablet PC 108b can be connected to one of the computers 104 and information can be synchronized. In an alternative embodiment, the mobile computing platform 108 can be in data communication with the server through a wireless network link. The mobile computing platform 108 also can be in data communication with a medical pump 110 through either a wired or a wireless link 112 such as a radio frequency (RF) data link or an infrared data link.

In an alternative embodiment, the medical pump 110 has a wireless network connector and can connect directly to the network 11 through a wireless link rather than through a computer 104 or a mobile computing platform 108. In yet another possible embodiment, the medical pump 110 could include a hardwired network connection.

The standing order database 114 includes a library of standing orders. Each standing order record includes information about the drug to which it relates, the therapy to which it relates, and a template for entering prescription information and pump operating parameters. The terms drug and medication are used interchangeably to refer to any fluid (e.g., pharmaceuticals, nutritional supplements, etc.) that a medical pump 110 might inject into a patient's body.

The database can include rules defining the data that a user can enter. For example, one rule might limit the identity of a drug to only certain types of therapies. Another rule might limit the range of pump operating parameters for a particular drug. Yet another rule might set predefined patient care instructions for a particular therapy or drug.

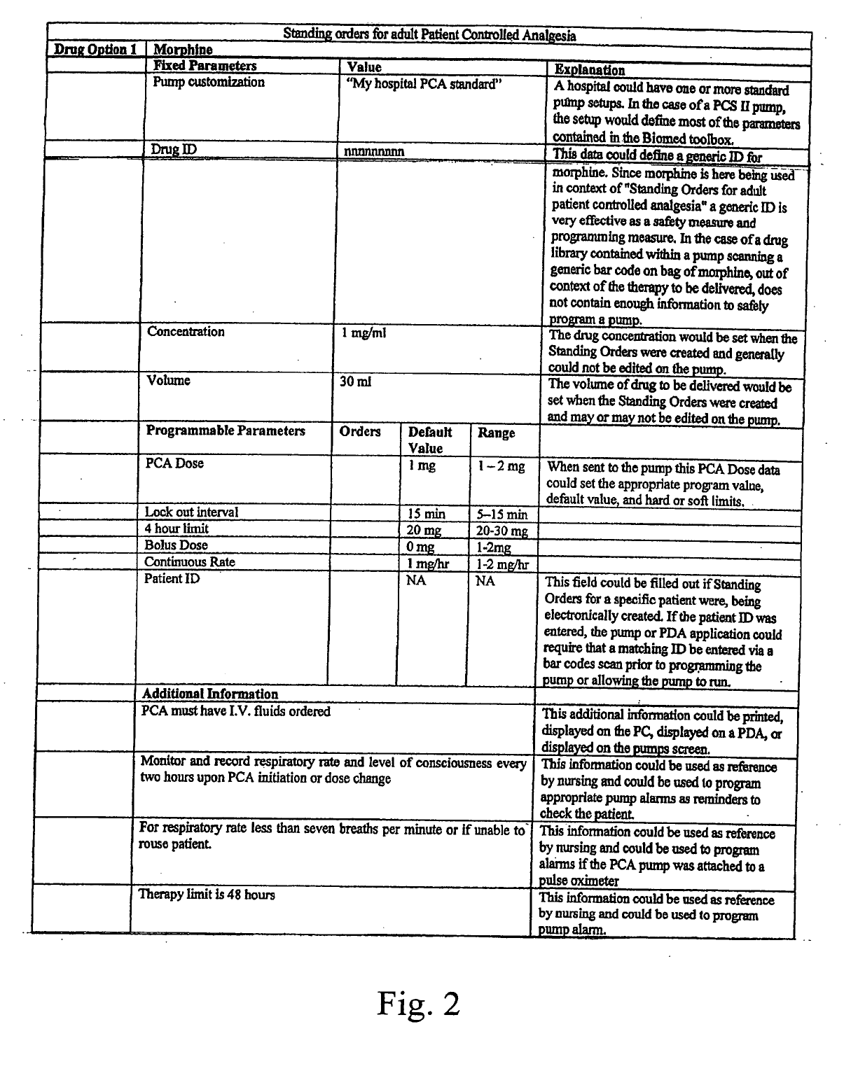

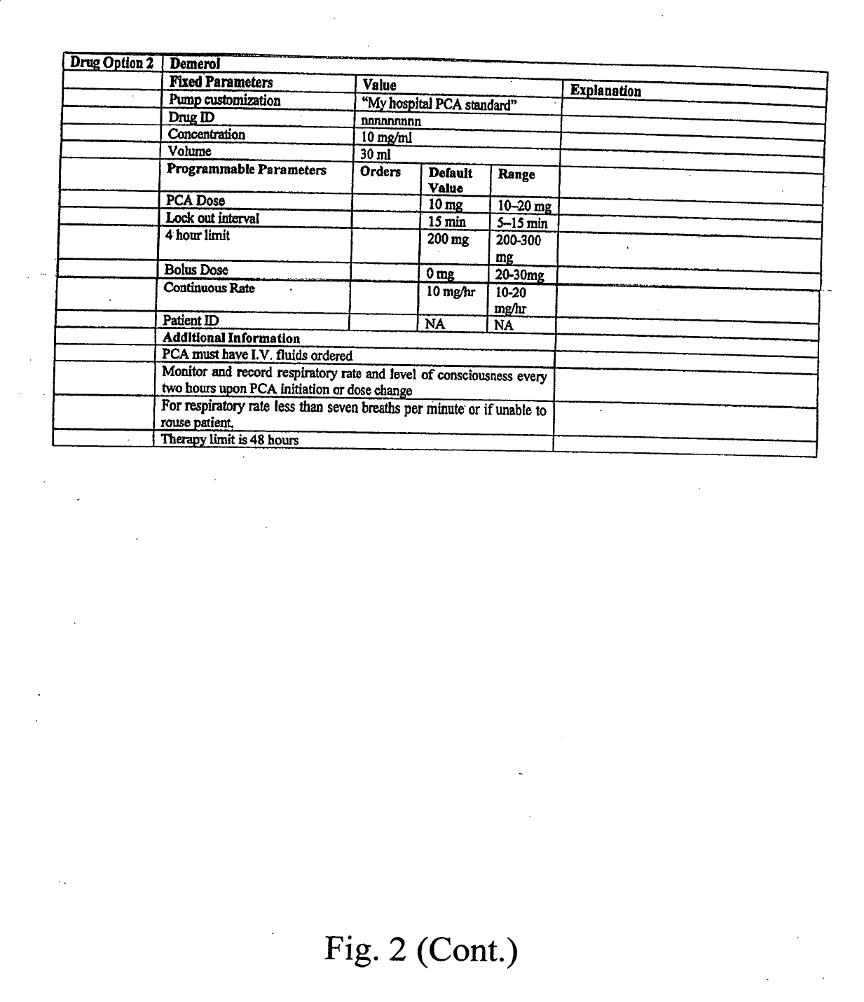

One possible embodiment of a standing orders template is shown in FIG. 2. Generally, the responsible person creates a standing order by naming it, for example “Standing Orders for adult patient controlled analgesia,” specifying the drug or drugs that can be used for this particular therapy, and specifying how the pump 110 should be programmed for this particular combination of therapy and drug. An advantage of this approach is that the drug is being used in context of the therapy specific standing order and therefore specific pump programming can be associated with the drug.

Once a library of electronic standing orders is created, the orders can be stored in the standing order database 114 and stored on an individual computer 104, stored on the server 102. Additionally, the library of electronic standing orders can be synchronized to a device such as a mobile computer 108, or downloaded into a medical pump 110. In other embodiments, the library of electronic standing orders can be stored on the pump 110. In yet other possible embodiments, only a smaller set of the electronic standing orders from the library of electronic standing orders is stored on the mobile computing platform 108 or the pump 110.

The patient database 116 includes a list of drugs and therapies prescribed to a particular patient. The patient database 116 includes a patient I.D. such as the patient name or identification number, patient care instructions specific to the patient, pump parameters, and a data key identifying the standing order template associated with the prescribed therapy and drug.

In an alternative embodiment, if the medical pump 110 is connected directly to the network 100 through a wireless link, the user retrieves the electronic standing order form directly on the pump 110 by connecting the pump 110 to the network 100 through a wireless network connection. The user can then directly access the library of electronic standing order forms on the database 106, select the desired electronic standing order form, and complete the electronic standing order form without having to synchronize the pump to a computer 104 or a mobile computing platform 108.

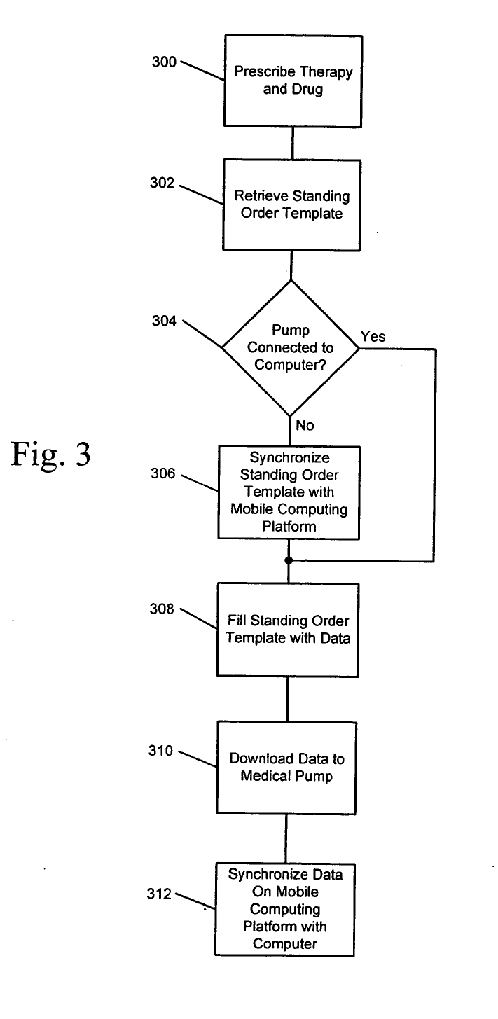

Referring to FIG. 3, in use a caregiver prescribes a particular therapy and associated drug for a patient at operation 300. The caregiver then retrieves the standing order template related to the prescribed therapy and drug at operation 302. In one possible embodiment, the caregiver first selects the prescribed therapy from a menu that lists the therapies for which there is an electronic standing order. The user then selects the prescribed drug from a submenu that is hierarchically subservient to the menu item corresponding to the selected therapy. The submenu of drugs will list those drugs that are available for use with the selected therapy. The user selects the prescribed drug from this list. In this embodiment, the various drugs that the pump 110 can deliver are organized and listed as subsets of the various therapies that use the drugs, and both the therapy and drugs are electronically linked to a particular electronic standing order.

The electronic standing order corresponding to the selected therapy and drug is produced for completion by the caregiver, operation 308. If the pump 110 is connected directly to a computer 104, the caregiver can fill in the standing order template, operation 308, on the computer 104 itself. If the pump 110 is not connected to a computer 104, the caregiver synchronizes the mobile computer with the database to download the standing order template, operation 306. The caregiver can then fill in the standing order template, operation 308, on the mobile computer 108. In another alternative embodiment, the mobile computing platform 108 is connected to the network 100 via a wireless network link and can retrieve the standing order template from the database 106 without being synchronized with a computer 104.

After a caregiver electronically completes the standing order template by filling out the “Orders” fields, operation 308, he or she saves the data of the standing order specific to a given patient. Patient data for the specific orders could then be found (perhaps by a scanning a patient's wristband), and sent to the pump 110, operation 310, and the patient can begin his or her therapy. The patient specific order is saved in the patient database 116. In one possible embodiment, the patient specific data is synchronized with the computer 104 and stored in the patient database 116, operation 312. In another possible embodiment, the patient specific data is not synchronized, but is stored in the patient database 116 through a wireless network connection between the mobile computer 108 and the server 102. Additionally, the “Orders” fields of a specific standing order/drug combination could be electronically filled out in advance by the physician or the nurse could fill in the blanks from written orders. The drug pump 110 could be programmed to require entries in all the “Orders” fields or be allowed to use default values.

Standing orders also typically include other instructions for patient care. For example, the standing order may include instructions on how often to evaluate the patient's condition or what to do if the patient's respiration drops below a certain threshold. Some of this additional instruction, if entered in a structured way, could be used by the pump 110 and/or other equipment to aid in patient care. For example, if the standing order instructs the evaluate nurse to evaluate and the patient's condition every two hours, the mobile computer 108 used to program the pump 110 or the pump 110 could sound an alarm every two hours as a reminder to the nurse. If the standing orders specify a respiratory rate below which the nurse is instructed to take action, the pump 110 could be combined with a pulse oximeter and using the data in the standing orders sound an alarm if respiration falls below specified limit.

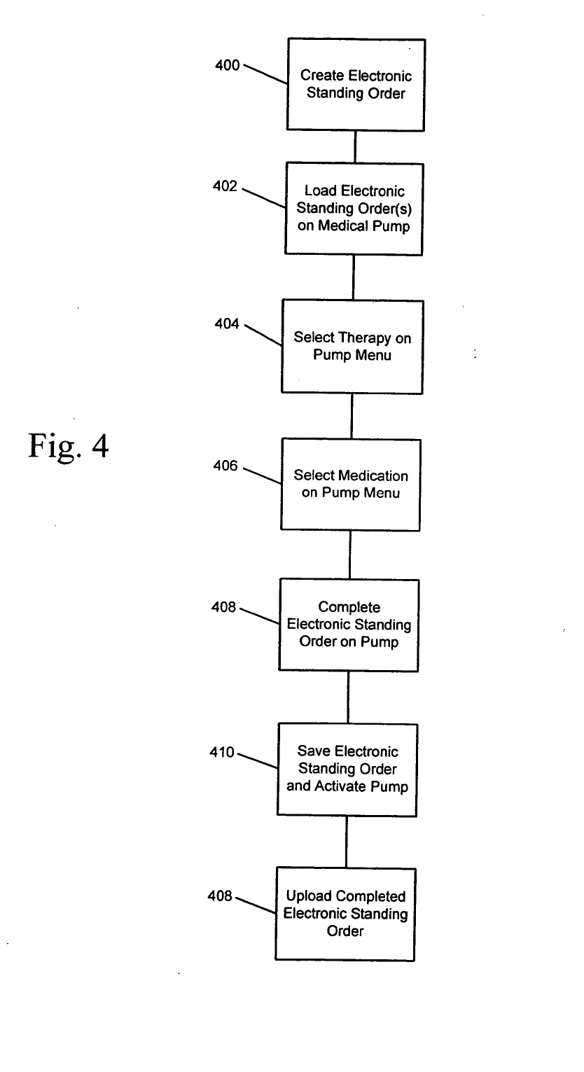

Yet another possible method of use is illustrated in FIG. 4. In this embodiment, the user creates one or more electronic standing order forms, operation 400, and stores one or more of the electronic standing order forms on the medical pump 100, operation 402. Once a therapy and drug are prescribed, the caregiver selects the prescribed therapy from a menu selection on the pump, operation 404, and then selects the prescribed drug, operation 406. The pump 110 then retrieves the electronic standing order that corresponds to the selected therapy and drug and the user completes the retrieved standing order form, operation 408. The user saves the completed electronic standing order form and activates the pump 110, operation 410. After the electronic standing order form is completed and activated, the pump 110 begins to deliver the drug using the delivery protocol set forth in the completed electronic standing order form. In one possible embodiment, the user can enter the patient's identification information into electronic standing order form so that it may be saved. One might store this information for historical purposes or so that the complete electronic standing order can be used to reprogram the pump 110 or a different pump 110 for that patient. The user also may upload the completed electronic standing order form and save it in the database 106.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention.

1. A computer readable medium, the computer readable medium in a computer apparatus, the computer readable medium containing a database, the database programmed to including a plurality of therapies, a plurality of therapeutic agents, at least one of the therapeutic agents being linked to at least one therapy, and a plurality of standing orders, each standing order including data for controlling operation of a programmable medical pump.

2. A computer readable medium of claim 1 wherein the computing apparatus is selected form the group consisting essentially of a hand-held computing apparatus, a tablet computing apparatus, and a desk-top computing apparatus.

3. A computer readable medium, the computer readable medium in a programmable medical pump, the computer readable medium containing a database, the database programmed to including a plurality of therapies, a plurality of therapeutic agents, at least one of the therapeutic agents being linked to at least one therapy, and a plurality of standing orders, each standing order including data for controlling operation of a programmable medical pump.
